(19) United States
(12) Patent Application Publication
(10) Pub. No.: US 2026/0123916 A1
(43) Pub. Date: May 7, 2026

(54) LESION CHARACTERIZATION APPARATUS AND METHOD USING 3D ULTRASOUND IMAGE

(71) Applicant: POSTECH RESEARCH AND BUSINESS DEVELOPMENT FOUNDATION, Pohang-si (KR)

(72) Inventors: Chul Hong KIM, Pohang-si (KR); Hyunhee KIM, Pohang-si (KR); Eunwoo PARK, Pohang-si (KR)

(73) Assignee: POSTECH RESEARCH AND BUSINESS DEVELOPMENT FOUNDATION, Pohang-si (KR)

(21) Appl. No.: 19/380,572

(22) Filed: Nov. 5, 2025

(30) Foreign Application Priority Data
Oct. 30, 2025 (KR) 10-2025-0160962
Nov. 6, 2024 (KR) 10-2024-0156065

(51) Int. Cl. A61B 8/00; A61B 8/08

(52) U.S. Cl. CPC A61B 8/483; A61B 8/085; A61B 8/466

(57) ABSTRACT

According to an aspect of the present disclosure, a lesion characterization apparatus using a three-dimensional (3D) ultrasound image includes a processor configured to set a 3D analysis region based on an ultrasound B-mode (brightness mode) image or an ultrasound C-scan (cross-section scan) image in the 3D ultrasound image, move the 3D analysis region, perform quantitative analysis based on lesion and peripheral signals included in the 3D analysis region, and reconstruct the 3D ultrasound image to highlight differences between lesion and peripheral signals by mapping quantitative values obtained through the analysis to respective voxels of the 3D analysis region. Unlike conventional 2D entropy imaging, the present disclosure applies a 3D window-based PDF estimation across spatial and frequency domains, providing voxel-wise entropy mapping.

### CROSS-REFERENCE TO RELATED APPLICATIONS

The present application claims priority under 35 U.S.C. § 119(a) to Korean patent applications number 10-2024-0156065 filed on November 6, 2024 and 10-2025-0160962 filed on October 30, 2025, in the Korean Intellectual Property Office, the entire disclosure of which is incorporated by reference herein.

### BACKGROUND

### 1. Technical Field

The present disclosure relates to a lesion characterization apparatus and a lesion characterization method using a three-dimensional (3D) ultrasound image.

### 2. Related Art

Conventional three-dimensional (3D) ultrasound imaging techniques mainly focus on providing structural images of lesions, and thus have limitations in effectively characterizing regions having physical properties different from those of a lesion or a peripheral region. Therefore, it is difficult to precisely analyze physical properties to detect a lesion at an early stage or to accurately identify abnormal tissues.

In addition, conventional ultrasound imaging technology can analyze physical properties only within an arbitrarily set region of interest (ROI), resulting in insufficient detailed analysis of the entire image. For example, ultrasound elastography requires a process of physically deforming tissue, necessitating additional equipment and procedures, which leads to a complicated analysis process and considerable time consumption. There is a quantitative ultrasound (qUS) method that analyzes using only conventional ultrasound B-mode; however, this method has the drawbacks of reduced resolution in analysis results and the inability to observe differences between the periphery region and the lesion in the image, providing only numerical values representing the overall tissue characteristics.

### SUMMARY

An object of the present disclosure is to provide a lesion characterization apparatus and a lesion characterization method for analyzing three-dimensional ultrasound signals to quantitatively and reproducibly characterize the physical properties of a lesion.

Another object of the present disclosure is to provide a lesion characterization apparatus and a lesion characterization method for precisely performing lesion characterization by combining probability density function–based and entropy analyses in both spatial and frequency domains, thereby simultaneously analyzing signal and frequency characteristics of a lesion.

A lesion characterization apparatus according to an embodiment of the present disclosure for solving the above technical problem includes: a storage configured to store at least one instruction; and a processor configured to execute the at least one instruction to perform analysis, wherein the processor is configured to record a three-dimensional (3D) ultrasound image in the storage, replace a first physical quantity for each center position of the 3D ultrasound image with a second physical quantity reflecting peripheral information of the center position to generate a reconstructed image mapped to each position, and provide lesion characterization information based on the reconstructed image.

In addition, a lesion characterization apparatus according to an embodiment of the present disclosure for solving the above technical problem includes a storage configured to store at least one instruction; and a processor configured to execute the at least one instruction to perform analysis, wherein the processor is configured to record an ultrasound image in the storage, generate a probability density function with respect to physical quantities for each center position of the ultrasound image and peripheral positions thereof, and record the probability density function in the storage, calculate a Shannon entropy for each center position based on the probability density function, generate a reconstructed image based on the Shannon entropy, and provide lesion characterization information based on the reconstructed image.

In an embodiment of the present disclosure, the processor may set a three-dimensional window defining the center position and a peripheral information region, and record information on the three-dimensional window in the storage, wherein the probability density function may represent a probability distribution of physical quantities corresponding to positions included in the three-dimensional window.

According to an embodiment of the present disclosure, the apparatus further includes a display unit, and the processor may display the reconstructed image on the display as an ultrasound B-mode (brightness mode) image or an ultrasound C-scan (cross-section scan) image format.

In an embodiment of the present disclosure, the processor may receive a plurality of B-mode ultrasound signals scanned in a direction perpendicular to a B-mode image surface, assign coordinates corresponding to the scanning direction to the respective B-mode ultrasound signals to generate the 3D ultrasound image.

In an embodiment of the present disclosure, the processor may receive a plurality of B-mode ultrasound signals scanned at predetermined time intervals, assign time-axis coordinates corresponding to the time intervals to the respective B-mode ultrasound signals to generate the 3D ultrasound image.

In an embodiment of the present disclosure, the processor may receive an ultrasound radio-frequency (RF) signal, and perform in-phase and quadrature (IQ) demodulation on the RF signal to generate first-stage processed data, perform beamforming on the first-stage processed data to generate second-stage processed data, and perform decimation on the second-stage processed data to generate an analysis reference image.

According to an embodiment of the present disclosure, the processor may receive an ultrasound RF signal, and perform beamforming on the RF signal to generate first-stage processed data, perform in-phase and quadrature (IQ) demodulation on the first-stage processed data to generate second-stage processed data, and perform decimation on the second-stage processed data to generate an analysis reference image.

In an embodiment of the present disclosure, the processor may perform singular value decomposition (SVD) on the 3D ultrasound image to generate an analysis reference image.

In an embodiment of the present disclosure, the processor may generate the probability density function for each center position and peripheral positions thereof using at least one of sound-velocity measurement, attenuation-coefficient analysis, Nakagami analysis, entropy analysis, scatterer-density analysis, and scatterer-size analysis, calculate the Shannon entropy in a frequency domain based on the probability density functions, derive the Shannon entropy for each frequency band, and calculate numerical values quantitatively evaluating similarity between a lesion region and a peripheral region based on the derived entropies, to generate the reconstructed image.

A lesion characterization method according to an embodiment of the present disclosure for solving the above technical problem includes recording an ultrasound image in a storage; generating a probability density function with respect to physical quantities for each center position of the ultrasound image and peripheral positions thereof, and recording the probability density function in the storage; calculating a Shannon entropy for each center position based on the probability density function; generating a reconstructed image based on the Shannon entropy; and providing lesion characterization information based on the reconstructed image.

According to an embodiment of the present disclosure, lesions such as cancer, cysts, nodules, and carotid artery stenosis may be accurately analyzed and diagnosed by utilizing the 3D ultrasound image, and physical characteristics of the lesions may be quantitatively evaluated and clearly distinguished from those of peripheral tissues.

According to an embodiment of the present disclosure, by analyzing a 3D ultrasound image before surgery, the position and the size of a lesion can be accurately identified to assist in establishing a surgical plan, or the present disclosure may be utilized to monitor changes in the lesion during treatment and evaluate therapeutic effects.

According to an embodiment of the present disclosure, since the present apparatus analyzes signals generated during the process of constructing an ultrasound image, a region different from a peripheral region can be characterized without a separate imaging system or process such as ultrasound elastography, thereby enabling lesions or surgical zones to be identified more simply.

### BRIEF DESCRIPTION OF THE DRAWINGS

FIG. 1 is a schematic diagram illustrating a lesion characterization system according to an embodiment of the present disclosure.

FIG. 2 is a block diagram illustrating a configuration of the lesion characterization apparatus 100 according to an embodiment of the present disclosure.

FIG. 3 is a flowchart illustrating a lesion characterization method according to an embodiment of the present disclosure.

FIGS. 4A and 4B are flowcharts illustrating a detailed step of a part of a lesion characterization method according to an embodiment of the present disclosure.

FIG. 5 is a flowchart illustrating a detailed step of a part of a lesion characterization method according to an embodiment of the present disclosure.

FIG. 6 is a conceptual diagram for explaining a 3D ultrasound image according to a first embodiment of the present disclosure.

FIG. 7 is a conceptual diagram for explaining a 3D ultrasound image according to a second embodiment of the present disclosure.

FIG. 8 is a conceptual diagram for explaining an analysis method according to a first embodiment of the present disclosure and a reconstructed image according thereto.

FIG. 9 is a conceptual diagram for explaining an analysis method according to a second embodiment of the present disclosure and a reconstructed image according thereto.

### DETAILED DESCRIPTION OF EMBODIMENT

This invention was made with support from the National Research and Development Program of Korea. The information of the supported project is as follows:

### Assignment Unique Number 2710014986

### Detailed Assignment Number 2021M3C1C3097624

### Name of the Ministry Korea Ministry of Science and ICT

### Research Management Specialized Organization National Research Foundation of Korea

### Research Project Title STEAM Research Program

### Assignment Title Development of a Cancer-Targeted Photoacoustic/Ultrasound Multimodal Imaging System

Name of the Organization Performing the Assignment POSTECH Research and Business Development Foundation

### Research Period January 1, 2024 – December 31, 2024

### Assignment Unique Number 234000473

### Detailed Assignment Number 2020R1A6A1A03047902

### Name of the Ministry Ministry of Education, Republic of Korea

### Research Management Specialized Organization National Research Foundation of Korea

### Research Project Title Basic Science Research Capacity Enhancement Program

### Assignment Title Medical Device Innovation Center

### Name of the Organization Performing the Assignment POSTECH Research and Business Development Foundation

### Research Period March 1, 2024 – February 28, 2025

Hereinafter, preferred embodiments of the present disclosure will be described in detail with reference to the accompanying drawings. The detailed description disclosed below, together with the accompanying drawings, is intended to explain exemplary embodiments of the present disclosure and is not intended to represent the only embodiments in which the present disclosure can be implemented. In the drawings, parts irrelevant to the description may be omitted to clearly explain the present disclosure, and the same reference numerals may be used for the same or similar components throughout the specification.

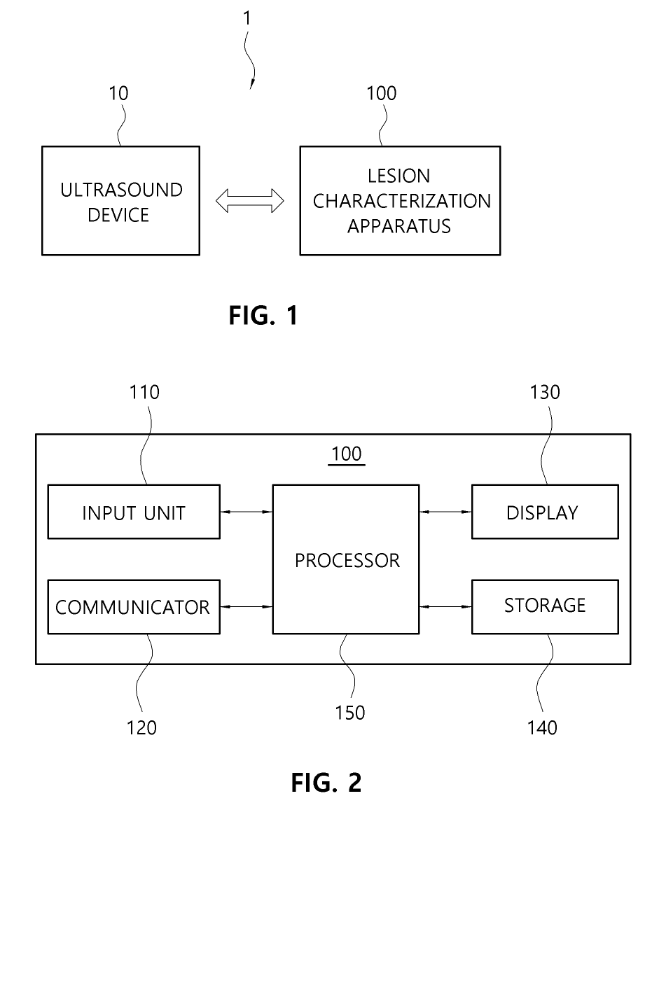

FIG. 1 is a schematic diagram illustrating a lesion characterization system according to an embodiment of the present disclosure.

The lesion characterization system according to an embodiment of the present disclosure includes an ultrasound device 10 and a lesion characterization apparatus 100.

The ultrasound device 10 applies an RF signal to a target to be analyzed and receives a signal reflected from the target. Specifically, the ultrasound device 10 may be a device that transmits an ultrasound signal to a target region and receives a reflected signal returning therefrom through a transducer.

The ultrasound device 10 may operate in a plurality of modes, for example, a basic B-mode (brightness mode) and a Doppler mode utilizing the Doppler effect. In this case, the Doppler mode includes a color Doppler mode, which may represent a flow direction of blood as a color and a flow velocity of blood as brightness, and a power Doppler mode, which may represent blood flow more sensitively than the color Doppler mode.

Various known ultrasound devices may be used as the ultrasound device described above, and thus detailed descriptions thereof will be omitted.

The ultrasound device 10 may transmit a reflected signal (RF signal) received by the lesion characterization apparatus 100.

The lesion characterization apparatus 100 is a computing device configured to analyze the reflected signal to derive lesion characteristics based on the 3D ultrasound image, and may be implemented as a computer, a server, a smartphone, a tablet PC, a smart pad, or a notebook computer.

The lesion characterization apparatus 100 may be connected to the ultrasound device 10 through a wired or wireless communication to receive a reflected signal. The reflected signal may be processed in real time and provided to a user as an image. However, in the present disclosure, a characteristic of the lesion may be more clearly represented through a 3D quantitative analysis of the reflected signal.

The ultrasound device 10 and the lesion characterization apparatus 100 may be implemented as a single integrated device (apparatus) or as two or more physically separated devices (apparatuses), and are not necessarily limited to any particular form or configuration.

The lesion characterization apparatus 100 constructs the 3D image from the RF signal and quantitatively analyzes the image with improved contrast-to-noise ratio and spatial precision, so that a specific location has physical properties different from those of a peripheral region.

Hereinafter, configurations and operations of the two devices according to an embodiment of the present disclosure will be described in detail with reference to the accompanying drawings.

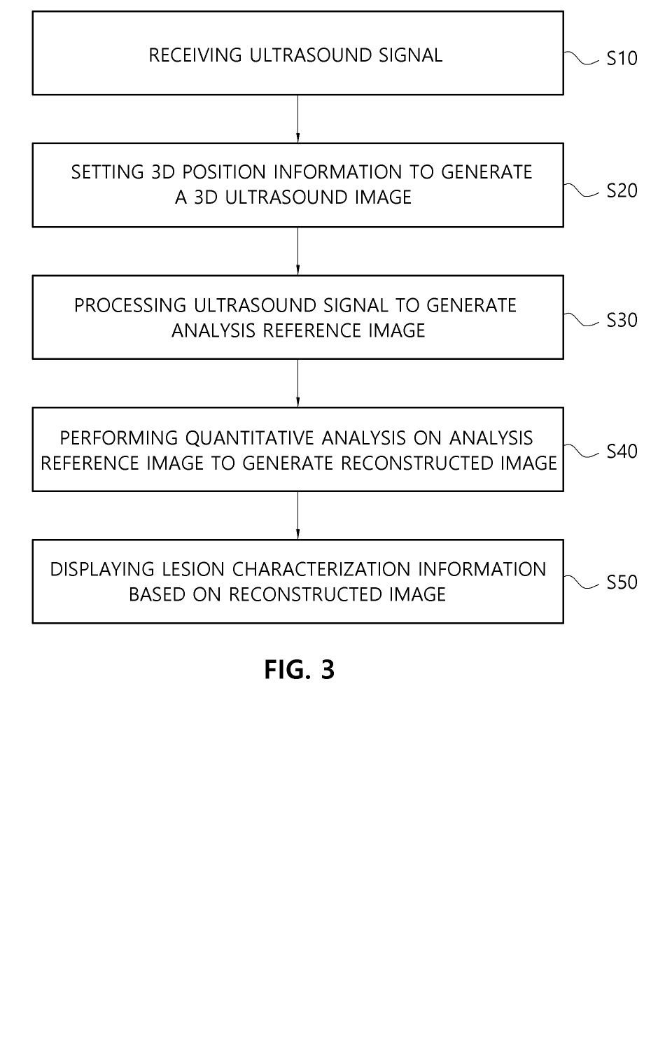

FIG. 2 is a block diagram illustrating a configuration of the lesion characterization apparatus 100 according to an embodiment of the present disclosure.

The lesion characterization apparatus 100 according to an embodiment of the present disclosure may include an input unit 110, a communicator 120, a display 130, a storage 140, and a processor.

The input unit 110 generates input data in response to a user input. For example, the user input may include an initiation of a processing operation, setting of data for constructing a 3D ultrasound image, setting of a 3D window 220 and a sliding path and range thereof, and selection of a quantitative analysis technique. In addition, the user input may include inputs for conditions and parameters required for operations of the lesion characterization apparatus 100, or for transmission paths of analysis results.

The input information may be provided to the processor or stored in the storage 140.

The input unit 110 may include at least one input means. The input unit 110 may include a keyboard, a keypad, a dome switch, a touch panel, a touch key, a mouse, or a menu button.

The communicator 120 may transmit and receive information with the ultrasound device 10, a server, and an external device.

The communicator 120 may perform wireless communication such as 5G (Fifth Generation Communication), LTE-A (Long Term Evolution-Advanced), LTE (Long Term Evolution), Wi-Fi (Wireless Fidelity), or Bluetooth.

The display 130 may visually present all information set or generated by the lesion characterization apparatus 100. Specifically, the display 130 may display a three-dimensional ultrasound image, a two-dimensional ultrasound image, a reconstructed image 250, or a user input.

The display 130 may include a liquid crystal display (LCD), a light emitting diode (LED) display, an organic light emitting diode (OLED) display, a microelectromechanical systems (MEMS) display, or an electronic paper display. The display 130 may be combined with the input unit 110 and implemented as a touch screen.

The storage 140 may store at least one instruction, including an operation program of the lesion characterization apparatus 100. The storage 140 may further store an analysis algorithm related to enabling the lesion characterization apparatus 100 to perform analysis. The analysis algorithm may include, for example, a method of setting a probability density function and a method of calculating a Shannon entropy.

The storage 140 includes non-volatile storage capable of retaining data (information) regardless of power supply, and volatile memory in which data to be processed by the processor is loaded, and which cannot retain data when power is not supplied. The storage may include, for example, a flash memory, a hard disk drive (HDD), a solid-state drive (SSD), or a read-only memory (ROM). The memory may include a buffer or a random access memory (RAM).

The storage 140 may store a two-dimensional ultrasound image, a 3D ultrasound image, a 3D analysis region, numerical values obtained through quantitative analysis, a reconstructed 3D ultrasound image, and an image visualizing a lesion. The storage 140 may also store operation programs required for processes such as generating the 3D ultrasound image, setting the 3D analysis region, performing quantitative analysis, reconstructing the 3D ultrasound image, and visualizing the lesion.

The processor may execute at least one instruction, program, or algorithm stored in the storage 140, receive an RF signal from the ultrasound device 10, and control the input unit 110, the communicator 120, the display 130, and the storage 140 to perform the lesion characterization method according to an embodiment of the present disclosure.

The processor may perform at least part of data analysis, processing, or generation of result information for executing the lesion characterization method by using at least one of a rule-based algorithm or an artificial intelligence (AI) algorithm, such as machine learning, a neural network, or a deep learning algorithm. Examples of the neural network may include models such as a convolutional neural network (CNN), a deep neural network (DNN), or a recurrent neural network (RNN).

In an embodiment of the present disclosure, the processor may set a 3D analysis region based on an ultrasound B-mode (brightness mode) image or an ultrasound C-scan (cross-section scan) image in the 3D ultrasound image, move the 3D analysis region, perform quantitative analysis based on lesion and peripheral signals included in the 3D analysis region, and reconstruct the 3D ultrasound image by mapping quantitative values obtained through the analysis to respective voxels of the 3D analysis region.

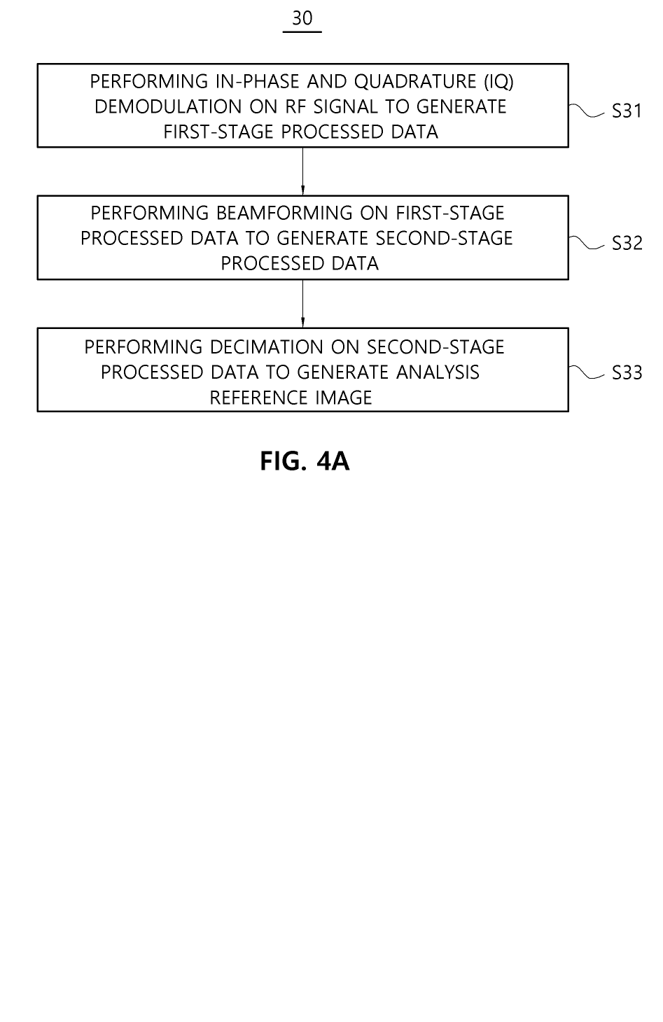

FIG. 3 is a flowchart illustrating a lesion characterization method according to an embodiment of the present disclosure. The lesion characterization method according to an embodiment of the present disclosure will be described as being executed by the lesion characterization apparatus 100 or a processor, but embodiments of the method are not necessarily limited thereto.

Referring to FIG. 3, the lesion characterization apparatus 100 receives an ultrasound signal (step S10). Here, the ultrasound signal refers to an RF signal that is transmitted by a transducer of the ultrasound device 10 and reflected from a target.

The processor generates a 3D ultrasound image by setting 3D positional information with respect to the received ultrasound signal (step S20).

The processor may analyze not only the 3D ultrasound image generated by itself, but also a 3D ultrasound image received from the ultrasound device 10 or an external device such as a server.

The 3D ultrasound image is information obtained by integrating collected two-dimensional data into information having 3D coordinates according to a predetermined criterion. The generation of the 3D ultrasound image may also be performed during processing the ultrasound signal to generate an analysis reference image 200 (step S30), in which the processing order is changed. The 3D ultrasound image information is recorded in the storage 140.

In the embodiment of the present disclosure, two criteria for generating a 3D ultrasound image are exemplified.

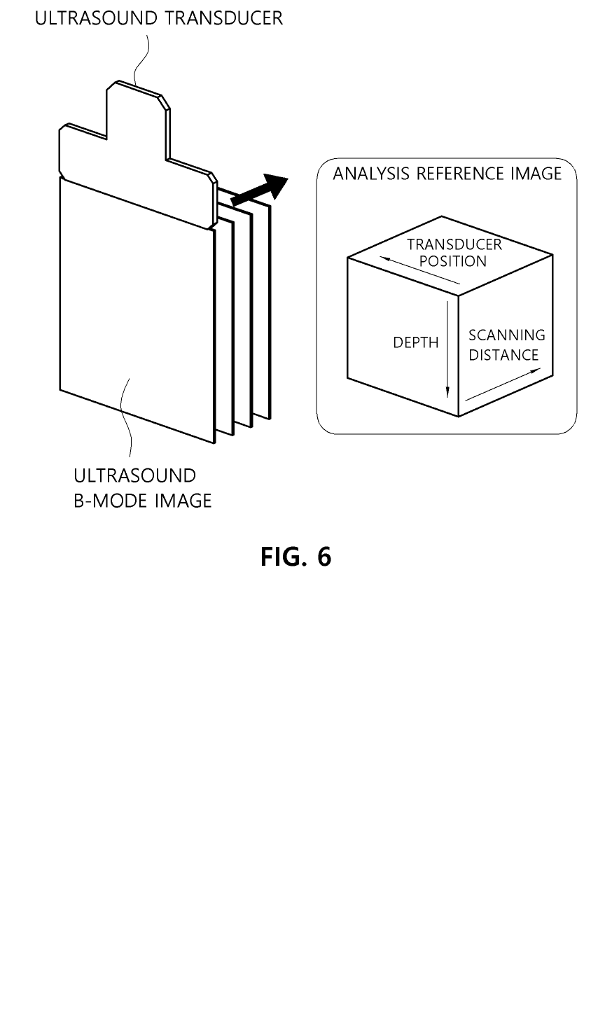

FIG. 6 is a conceptual diagram for explaining a 3D ultrasound image according to a first embodiment of the present disclosure.

The 3D ultrasound image according to the first embodiment may use a B-mode image 230, which is two-dimensional data distributed in a plane. The B-mode image 230 is a two-dimensional image composed of a direction (depth direction) in which a transducer transmits and receives ultrasound, and a width direction of the transducer.

The target may be scanned by the ultrasound transducer in a direction perpendicular to the B-mode image 230. In this case, a plurality of B-mode images 230 may be obtained along another one-dimensional axis. The 3D ultrasound image information may be generated by integrating the positional information of the one-dimensional axis into the two-dimensional B-mode images 230.

The 3D ultrasound image according to the first embodiment is suitable for analyzing a spatial distribution of a lesion at a specific point in time.

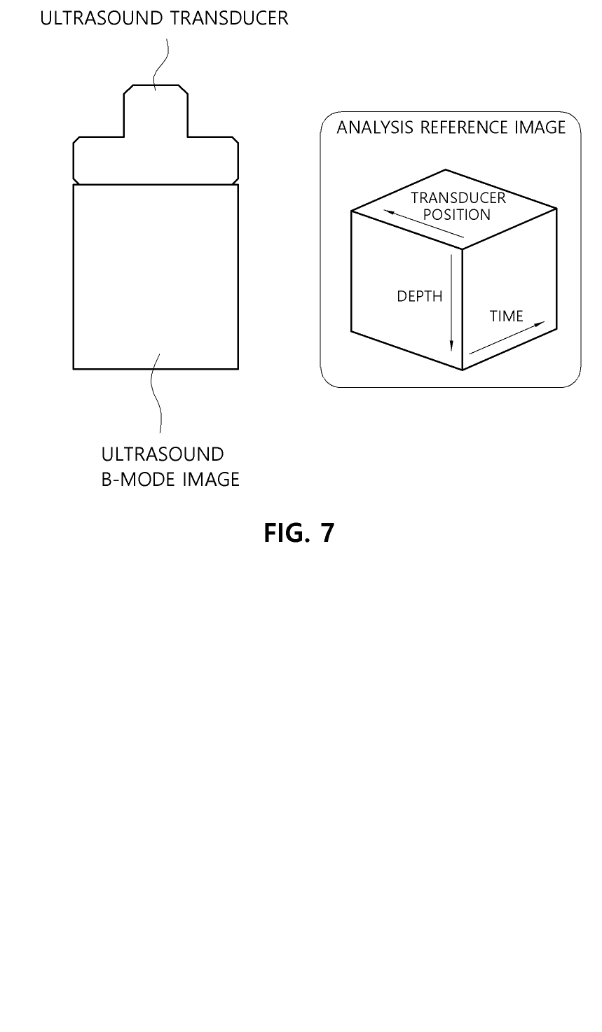

FIG. 7 is a conceptual diagram for explaining a 3D ultrasound image according to a second embodiment of the present disclosure.

The 3D ultrasound image according to the second embodiment may use a B-mode image 230, which is two-dimensional data distributed in a plane.

In the second embodiment, a position of the ultrasound transducer is not changed, and a plurality of B-mode images 230 are acquired at predetermined time intervals. The 3D ultrasound image information may be generated by integrating one-dimensional time information into the two-dimensional B-mode images 230.

The 3D ultrasound image according to the second embodiment is more suitable for analyzing a temporal change of the lesion rather than the current distribution of the lesion.

The processor processes the ultrasound signal to generate an analysis reference image 200 (step S30). In this case, the ultrasound signal may be a signal that has already been constructed in 3D or a signal that has not yet been constructed in 3D.

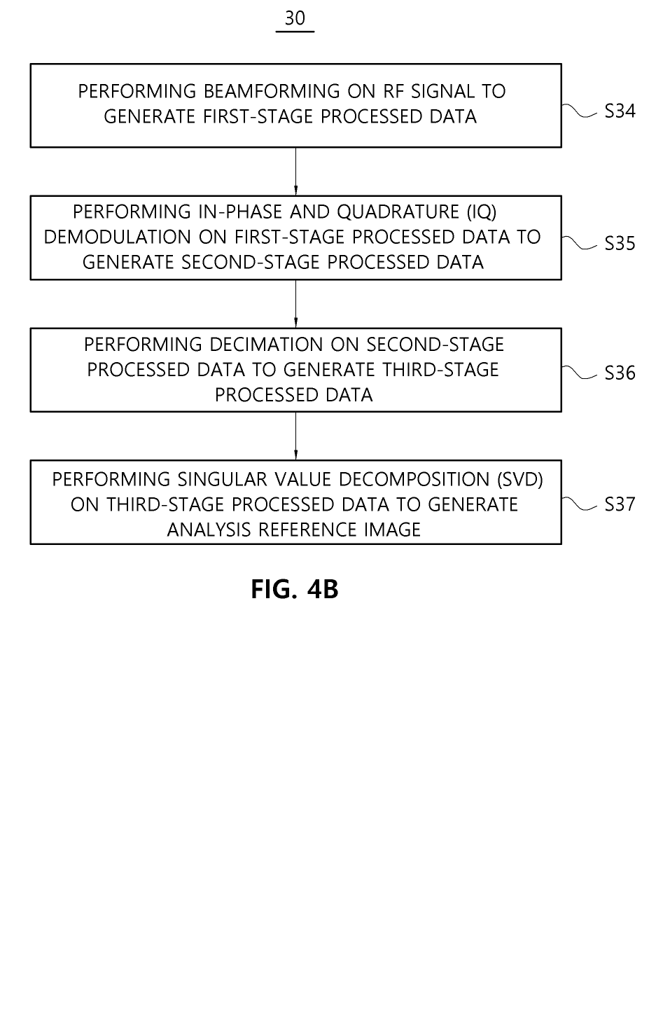

FIGS. 4A and 4B are flowcharts illustrating step S30, which details a method of processing an ultrasound signal to generate an analysis reference image 200.

FIG. 4A shows a method of generating the analysis reference image 200 according to a first embodiment. Referring to FIG. 4A, the processor performs in-phase and quadrature (IQ) demodulation on an RF signal to generate first-stage processed data (step S31).

The IQ demodulation is a preprocessing operation that decomposes an original signal into cosine and sine components and converts it into a baseband signal. Performing the IQ demodulation enables stable extraction of phase and amplitude components of the signal, reduces distortion and unnecessary amounts of computation caused by high-frequency components during beamforming or entropy analysis, and suppresses noise when extracting a local probability density function.

The processor performs beamforming on the first-stage processed data to generate second-stage processed data (step S32). Performing beamforming after IQ demodulation is referred to as baseband beamforming.

Beamforming is a process of compensating for time delays among reflected signals received by respective ultrasound sensors and summing the signals, which is also referred to as a delay-and-sum operation.

The processor performs decimation on the second-stage processed data to generate the analysis reference image 200 (step S33).

The decimation is an operation of lowering a sampling frequency to improve efficiency in signal processing.

The analysis reference image 200 refers to a 3D ultrasound image on which data processing, including signal processing and preprocessing of the RF signal, has been performed. As described above, the setting of 3D positional information may be executed before step S31, between steps S31 and S32, or between steps S32 and S33.

FIG. 4B shows a method of generating an analysis reference image 200 according to a second embodiment of the present disclosure. Referring to FIG. 4B, the processor performs beamforming on the RF signal to generate first-stage processed data (step S34). Such beamforming is referred to as delay-and-sum beamforming.

The processor performs in-phase and quadrature (IQ) demodulation on the first-stage processed data to generate second-stage processed data (step S35).

The order of IQ demodulation and beamforming may vary depending on system configuration; both orders are encompassed within the present disclosure.

The processor may perform decimation on the second-stage processed data to generate the analysis reference image 200 (step S36). However, in the second embodiment, the processor may perform Singular Value Decomposition (SVD) on third-stage processed data, which is obtained by performing decimation on the second-stage processed data, to generate the analysis reference image 200 (step S37). Here, the analysis reference image 200 refers to a 3D ultrasound image that has sequentially undergone beamforming, IQ demodulation, decimation, and SVD.

The SVD is a mathematical decomposition method that reconstructs a signal into a matrix and separates noise components, tissue components, and speckle variation components. In the present disclosure, the SVD is applied to remove noise components and selectively extract temporal and spatial signal variations generated from a lesion, thereby improving the accuracy of entropy analysis.

The SVD may be performed on a 3D ultrasound image constructed by the method of FIG. 7, which includes time-axis information. Accordingly, the SVD may also be applied to the method of generating the analysis reference image 200 according to the first embodiment.

Meanwhile, when time-axis information is not included, the SVD may be omitted.

The processor performs quantitative analysis on the analysis reference image 200 to generate a reconstructed image 250 (step S40).

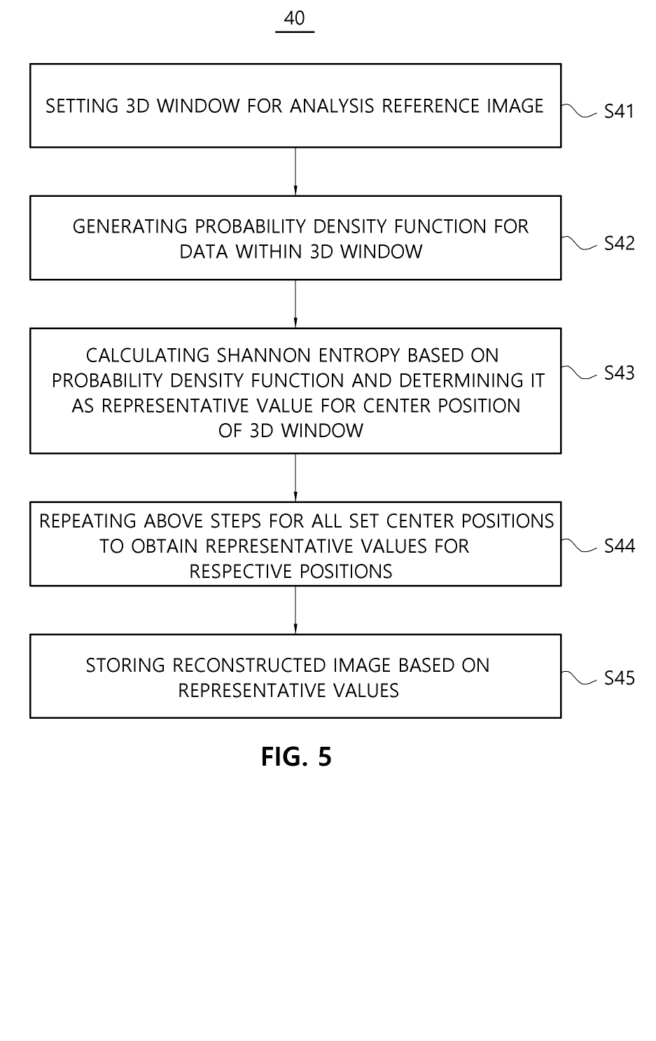

FIG. 5 is a flowchart illustrating step S40, which details a method of performing quantitative analysis on the analysis reference image 200 to generate the reconstructed image 250.

Referring to FIG. 5, the processor sets a 3D window 220 for the analysis reference image 200 and records the same in the storage 140 (step S41).

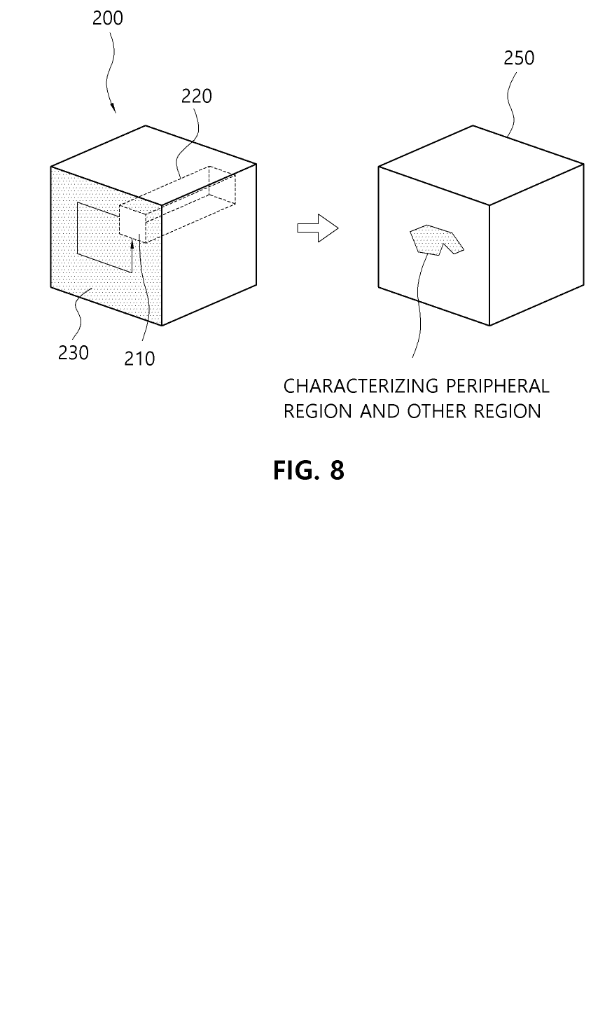

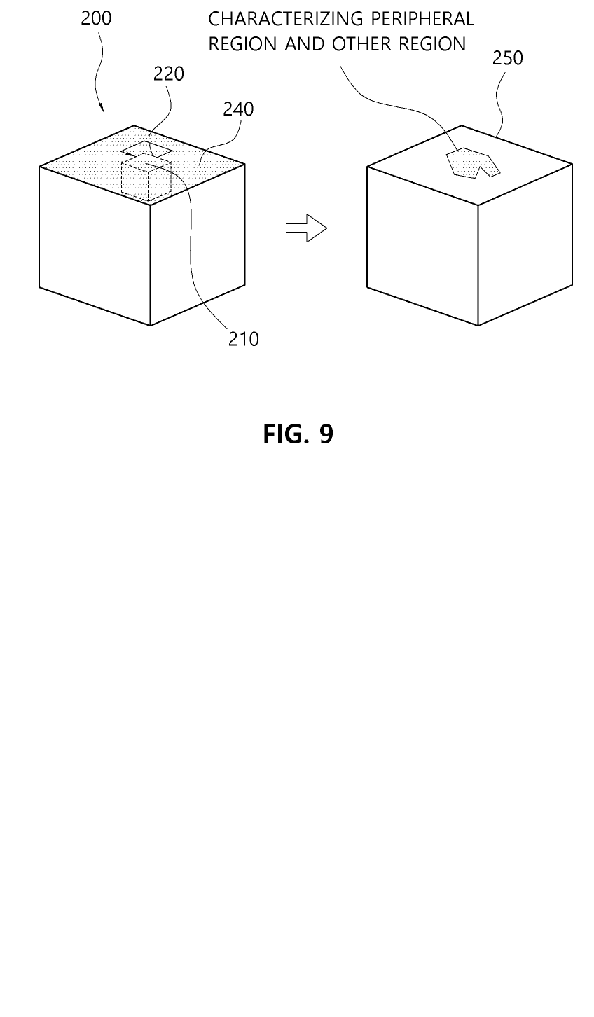

The 3D window 220 may be a selected partial region of all data of the 3D ultrasound image. For example, the 3D window 220 may be selected as a rectangular-parallelepiped region (see FIG. 8) or as a cubic region (see FIG. 9).

The 3D window 220 may be selected based on a center position 210. The center position 210 may be a 3D coordinate (which may be a voxel unit) on which a result value obtained by analyzing a region corresponding to the 3D window 220 is to be mapped.

The 3D window 220 may be selected to derive a result value by incorporating information from a peripheral region of the center position 210 together when analyzing the center position 210.

The 3D window 220 may be selected as a region within a predetermined range centered on the center position 210. However, it is not necessarily required that the center position 210 always be located at a central coordinate of the 3D window 220, and an appropriate region having various positional relationships with the center position 210 may be selected as needed.

The processor generates a probability density function (PDF) for data within the 3D window 220 (step S42).

The probability density function is a normalized function representing how frequently a physical quantity indicated by the data within the 3D window 220 appears.

The probability density function (PDF) may be computed by histogramming voxel-wise signal amplitudes or by kernel density estimation applied to intensity values within the 3D window.

For example, the probability density function may represent a probability distribution of an amplitude of the ultrasound signal. In relation to lesion characteristics, distributions of the probability density function in a normal region and a peripheral region around the normal region are expected to be similar, whereas distributions of the probability density function in a lesion region and a peripheral region around the lesion are different, and the degree of variation in the difference is large.

In the present disclosure, a Shannon entropy (e.g., Shannon Differential Entropy, SDE) is used for the probability density function to quantitatively observe variations in the probability density function.

The Shannon entropy is a quantitative value representing complexity or inhomogeneity of a signal. The Shannon entropy value increases as randomness of the signal increases, and decreases as the pattern of the signal becomes more consistent.

The processor calculates the Shannon entropy based on the probability density function and determines the calculated Shannon entropy as a representative value for the center position 210 (step S43).

The Shannon entropy is a measure capable of evaluating the degree of disorder of a distribution of the probability density function, and has a characteristic in which relative relationships between physical quantities at a corresponding voxel coordinate and peripheral physical quantities are reflected instead of an absolute physical quantity at the voxel coordinate.

Accordingly, when the coordinates of the 3D ultrasound image are reconstructed based on the Shannon entropy, characteristics appearing at each coordinate become more distinct.

The processor repeats setting the 3D window (step S41), generating the probability density function (step S42), and calculating the Shannon entropy (step S43) for other center positions 210, thereby calculating the Shannon entropy for all voxel coordinates and storing the same in the storage 140 (step S44).

The processor maps data generates a 3D reconstructed image by mapping representative value based on Shannon entropy to each corresponding coordinate, thereby generating a 3D reconstructed image and storing the same in the storage 140 (step S45).

In the present disclosure, the term ‘reconstructed image’ refers to a three-dimensional map in which each value corresponds to a similarity value that is visualized as an entropy-based contrast image.

The processor displays lesion characterization information based on the reconstructed image 250 (step S50).

The lesion characterization information may be displayed as a B-mode image 230 or a C-scan image 240, which is two-dimensionally extracted from a portion of the reconstructed image 250 that represents the reconstructed image.

FIG. 8 is a conceptual diagram for explaining an analysis method according to a first embodiment of the present disclosure and a reconstructed image according thereto.

In FIG. 8, a 3D window 220 set in a rectangular-parallelepiped shape and a corresponding center position 210 for the analysis reference image 200 are shown (left side), and the reconstructed image 250 schematically illustrates that characteristics of a lesion are distinctly reflected (right side).

A shaded two-dimensional plane may represent the B-mode image 230.

In FIG. 8, sliding of the 3D window 220 in a counterclockwise direction is illustrated. However, the sliding direction of the 3D window 220 is not limited to this embodiment, as long as all regions to be analyzed are covered.

FIG. 9 is a conceptual diagram for explaining an analysis method according to a second embodiment of the present disclosure and a reconstructed image according thereto.

In FIG. 9, a 3D window 220 set in a cubic shape and a corresponding center position 210 for the analysis reference image 200 are shown (left side), and the reconstructed image 250 schematically illustrates that characteristics of a lesion are distinctly reflected (right side).

A shaded two-dimensional plane may represent the C-scan image 240.

The present disclosure relates to analyzing characteristics of a lesion based on an ultrasound signal, and a lesion may exhibit physical characteristics quantitatively different from those of a peripheral region. For example, a region in which protein denaturation occurs due to surgery, a region where temperature changes, or a lesion such as cancer, a cyst, or a nodule has physical characteristics different from those of a peripheral region thereof.

Meanwhile, a spatiotemporally accumulated 3D ultrasound image may be utilized to observe changes in physical characteristics of a lesion during surgery.

In an embodiment of the present disclosure, the probability density function (PDF) is obtained with respect to an intensity of an ultrasound signal, but is not necessarily limited thereto. For example, the processor may generate a probability density function for a physical quantity obtained as a quantitative analysis result by using at least one of sound velocity measurement, attenuation-coefficient analysis, Nakagami analysis, entropy analysis, scatterer-density analysis, and scatterer-size analysis, and may calculate a Shannon entropy from the probability density function. The Shannon entropy may quantitatively reflect a difference in a signal distribution between a specific position and a peripheral region thereof.

The processor may move the 3D analysis region voxel by voxel based on the ultrasound B-mode image or the ultrasound C-scan image. In this case, the movement path of the 3D analysis region is not limited to any particular path, as long as analysis may be performed for all voxels of the 3D ultrasound image.

The processor may reconstruct the 3D ultrasound image by mapping quantitative analysis values to respective voxels of the 3D analysis region.

According to an embodiment of the present disclosure, lesions such as cancer, cysts, nodules, and carotid artery stenosis may be accurately analyzed and diagnosed by utilizing the 3D ultrasound image, and physical characteristics of the lesions may be quantitatively evaluated and clearly distinguished from those of peripheral tissues.

According to an embodiment of the present disclosure, by analyzing a 3D ultrasound image before surgery, a position and a size of a lesion can be accurately identified to assist in establishing a surgical plan, or the present disclosure may be utilized to monitor changes in the lesion during treatment and evaluate therapeutic effects.

The processor may control a display 130 to visually present analysis results. The processor may provide an image in a B-mode or C-scan format based on mapped values of the reconstructed image 250.

The processor may control the display 130 to make a lesion more prominent from a peripheral region by mapping numerical values to corresponding colors.

The processor may display a reconstructed B-mode image of the reconstructed image 250 layered on a B-mode image among 3D ultrasound images. In this case, the reconstructed B-mode image may be a two-dimensional ultrasound image of a section of the reconstructed 3D ultrasound image in which a lesion is clearly visible, but is not limited thereto.

According to an embodiment of the present disclosure, a difference in physical properties of specific tissues may be quantitatively analyzed through an ultrasound image to assist in early detection of lesions such as tumors. In addition, accurate positional information may be provided through the 3D image to offer useful information for establishing a surgical plan and during a treatment process.

According to an embodiment of the present disclosure, since analysis is performed based on an ultrasound signal, a region having physical characteristics different from those of a peripheral region may be characterized, which may include a region in which protein denaturation occurs due to a surgical instrument, a region where temperature changes, and a lesion such as cancer, a cyst, or a nodule.

The processor may calculate a probability density function of a signal intensity within each 3D analysis region in spatial and frequency domains to evaluate distribution characteristics of a lesion signal and a peripheral signal. In the frequency domain, the processor may decompose the signal into frequency components through a Fourier transform and estimate a probability density function of a specific frequency band to identify frequency characteristics and an irregularity of a lesion.

The processor may calculate a probability density function of a frequency band through a histogram or kernel density estimation to simultaneously reflect frequency and spatial characteristics of a signal.

The processor may calculate a Shannon entropy in the frequency domain based on the obtained probability density function.

Through this, the processor may quantify the uncertainty and randomness of the signal. By calculating a Shannon entropy from the probability density function, the processor may distinguish frequency characteristics between the inside of a lesion and peripheral tissues, and perform an operation of clearly visualizing or quantifying characteristics such as differences in signal distribution.

The processor may calculate a value that quantitatively evaluates similarity between the lesion and the peripheral region by deriving a Shannon entropy for each frequency band.

The processor may compare frequency characteristics between the lesion region and the peripheral region, such as a tissue, by deriving an entropy value for each frequency band, which may effectively represent a difference in signal distribution between cystic and solid lesions.

Through frequency-based entropy analysis, an irregularity emphasized by frequency characteristics of the lesion may be identified, thereby making lesion characterization more precise.

Subsequently, the processor may quantitatively evaluate a similarity between the lesion signal and the peripheral signal based on the entropy value calculated in spatial and frequency domains, and map the evaluated similarity to pixels of each analysis region.

The evaluation of the similarity may be performed by quantitatively comparing a pattern difference between the lesion core and the normal tissue using an SDE-based Shannon entropy value.

Specifically, after the Shannon entropy is calculated for each voxel, a distribution difference between the estimated lesion region and the peripheral region, such as healthy tissue or a margin, is converted into a similarity or difference metric. The similarity may be quantified for each voxel and visually represented as a heatmap overlay to make the boundary of the lesion more clearly visible.

If the Shannon entropy quantifies the complexity of each voxel, the similarity evaluation corresponds to post-processing that compares the quantified values between groups, that is, between the lesion region and the peripheral region, to enhance a lesion boundary and visual contrast.

Through this, the distribution and boundary of the lesion in the 3D image may be clearly visualized and reconstructed, thereby enabling clearer diagnostic visualization of lesion boundaries. With the addition of the frequency-domain entropy analysis, not only structural characteristics of the lesion but also frequency characteristics are reflected, providing a precise lesion visualization.

What is claimed is:

1. A lesion characterization apparatus comprising:
a storage configured to store at least one instruction; and
a processor configured to execute the at least one instruction to perform analysis,
wherein the processor is configured to:
record a three-dimensional (3D) ultrasound image in the storage,
replace a first physical quantity for each center position of the 3D ultrasound image with a second physical quantity reflecting peripheral information of the center position to generate a reconstructed image mapped to each position, and
provide lesion characterization information based on the reconstructed image.

2. A lesion characterization apparatus comprising:
a storage configured to store at least one instruction; and
a processor configured to execute the at least one instruction to perform analysis,
wherein the processor is configured to:
record an ultrasound image in the storage,
generate a probability density function with respect to physical quantities for each center position of the ultrasound image and peripheral positions thereof, and record the probability density function in the storage,
calculate a Shannon entropy for each center position based on the probability density function,
generate a reconstructed image based on the Shannon entropy, and
provide lesion characterization information based on the reconstructed image.

3. The lesion characterization apparatus of claim 2, wherein the processor is configured to set a three-dimensional window defining the center position and a peripheral information region, and record information on the three-dimensional window in the storage, and wherein the probability density function represents a probability distribution of physical quantities corresponding to positions included in the three-dimensional window.

4. The lesion characterization apparatus of claim 1, further comprising a display,
wherein the processor is configured to display the reconstructed image on the display as an ultrasound B-mode (brightness mode) image or an ultrasound C-scan (cross-section scan) image format.

5. The lesion characterization apparatus of claim 1, wherein the processor is configured to:
receive a plurality of B-mode ultrasound signals scanned in a direction perpendicular to a B-mode image surface,
assign coordinates corresponding to the scanning direction to the respective B-mode ultrasound signals to generate the 3D ultrasound image.

6. The lesion characterization apparatus of claim 1, wherein the processor is configured to:
receive a plurality of B-mode ultrasound signals scanned at predetermined time intervals,
assign time-axis coordinates corresponding to the time intervals to the respective B-mode ultrasound signals to generate the 3D ultrasound image.

7. The lesion characterization apparatus of claim 1, wherein the processor is configured to:
receive an ultrasound radio-frequency (RF) signal, and perform in-phase and quadrature (IQ) demodulation on the RF signal to generate first-stage processed data,
perform beamforming on the first-stage processed data to generate second-stage processed data, and
perform decimation on the second-stage processed data to generate an analysis reference image.

8. The lesion characterization apparatus of claim 1, wherein the processor is configured to:
receive an ultrasound RF signal, and perform beamforming on the RF signal to generate first-stage processed data,
perform in-phase and quadrature (IQ) demodulation on the first-stage processed data to generate second-stage processed data, and
perform decimation on the second-stage processed data to generate an analysis reference image.

9. The lesion characterization apparatus of claim 6, wherein the processor is configured to perform singular value decomposition (SVD) on the 3D ultrasound image to generate an analysis reference image.

10. The lesion characterization apparatus of claim 2, wherein the processor is configured to:
generate the probability density function for each center position and peripheral positions thereof using at least one of sound-velocity measurement, attenuation-coefficient analysis, Nakagami analysis, entropy analysis, scatterer-density analysis, and scatterer-size analysis,
calculate the Shannon entropy in a frequency domain based on the probability density functions,
derive the Shannon entropy for each frequency band, and
calculate numerical values quantitatively evaluating similarity between a lesion region and a peripheral region based on the derived entropies, to generate the reconstructed image.

11. A lesion characterization method performed by a lesion characterization apparatus, the method comprising:
recording an ultrasound image in a storage;
generating a probability density function with respect to physical quantities for each center position of the ultrasound image and peripheral positions thereof, and recording the probability density function in the storage;
calculating a Shannon entropy for each center position based on the probability density function;
generating a reconstructed image based on the Shannon entropy; and
providing lesion characterization information based on the reconstructed image.
